(19) United States
(12) Patent Application Publication
(10) Pub. No.: US 2025/0151989 A1
(43) Pub. Date: May 15, 2025

(54) ENDOSCOPE IMAGING DEVICE, ENDOSCOPE DEVICE, AND ATTACHMENT DEVICE

(71) Applicant: Sony Olympus Medical Solutions Inc., Tokyo (JP)

(72) Inventor: Fuminori TANAHASHI, Tokyo (JP)

(73) Assignee: Sony Olympus Medical Solutions Inc., Tokyo (JP)

(21) Appl. No.: 18/925,057

(22) Filed: Oct. 24, 2024

(30) Foreign Application Priority Data
Nov. 13, 2023 (JP) 2023-192813

(51) Int. Cl. A61B 1/00

(52) U.S. Cl. CPC A61B 1/00195; A61B 1/00105

(57) ABSTRACT

An endoscope imaging device that includes an attachment portion detachably connected to an eyepiece portion of an endoscope and captures a subject image emitted from the eyepiece portion, the endoscope imaging device including: a first portion of metal having a first sliding surface; a second portion of metal having a second sliding surface that slides on the first sliding surface; and a resin portion configured on at least one of the first sliding surface or the second sliding surface.

### CROSS-REFERENCE TO RELATED APPLICATION

This application claims the benefit of Japanese Priority Patent Application JP 2023-192813 filed on Nov. 13, 2023, the entire contents of which are incorporated herein by reference.

### TECHNICAL FIELD

The present disclosure relates to an endoscope imaging device, an endoscope device, and an attachment device.

### BACKGROUND ART

Conventionally, an endoscope device that observes the inside of a subject such as a person or a mechanical structure is known in a medical field or an industrial field. Such an endoscope device includes an endoscope that captures a subject image in a subject and emits the subject image from an eyepiece portion, and an endoscope imaging device that includes an attachment portion (coupler unit) to which the eyepiece portion is detachably connected and captures the subject image emitted from the eyepiece portion.

### CITATION LIST

### Patent Literature

[PTL 1]
JP 2022-145015 A
[PTL 2]
WO 2016/103805

### SUMMARY

### Technical Problem

However, there is a possibility that metal abrasion powder is generated on the sliding surfaces when components that slide between metals due to a rotational operation are included and sliding is repeated.

Therefore, the present disclosure provides an endoscope imaging device, an endoscope device, and an attachment device capable of suppressing abrasion between components sliding on each other.

### Solution to Problem

In order to solve the above problems, according to the present disclosure, there is provided an endoscope imaging device that includes an attachment portion detachably connected to an eyepiece portion of an endoscope and captures a subject image emitted from the eyepiece portion, the endoscope imaging device including:
a first portion of metal having a first sliding surface;
a second portion of metal having a second sliding surface that slides on the first sliding surface; and
a resin portion configured on at least one of the first sliding surface and the second sliding surface.

The second portion may be configured to rotate or be rotatable with respect to a predetermined rotation axis.

The predetermined rotation axis may be an optical axis of the endoscope or an axis parallel to the optical axis of the endoscope.

The resin portion may be formed in a hole formed in at least one of the first sliding surface and the second sliding surface.

An adhesive may be filled between the hole and the resin portion.

The attachment portion may include:
the first portion; and
the second portion.

The attachment portion may include: a mount that holds the endoscope with a locking member; and a ring that is provided on an outer periphery of the mount and is capable of moving the locking member into the mount in accordance with the rotation, and the first portion may be the mount, and the second portion may be the ring.

At least a part of an outer peripheral surface of the mount may be the first sliding surface, and at least a part of an inner peripheral surface of the ring may be the second sliding surface.

A distance from the rotation axis to an end portion of the resin portion along the inner peripheral surface of the ring may be larger than a distance from the rotation axis to the outer peripheral surface of the mount and shorter than a distance from the rotation axis to an end portion along the inner peripheral surface of the ring. The mount may include a protrusion disposed along an outer periphery of the mount and having a first side surface orthogonal to the rotation axis,
the ring may include a movement prevention portion disposed along an inner peripheral surface of the ring and having a second side surface orthogonal to the rotation axis and sliding on the first side surface, and at least a part of the first side surface may be the first sliding surface, and at least a part of the second side surface may be the second sliding surface.

The attachment portion may be rotatably attached to an outer periphery of an opening of a main body of the endoscope imaging device, and the first portion may be the attachment portion, and the second portion may be the main body.

The attachment portion may include an annular portion formed at an equal distance from a predetermined rotation axis,
the main body may include a clamping portion that clamps the annular portion, and
the resin portion may be formed on a sliding surface of at least one of the annular portion and the clamping portion.

The clamping portion may clamp the annular portion with three concave surfaces, and
the resin portion may be configured on at least one of the three surfaces.

The metal may be at least one of aluminum, an aluminum alloy, stainless steel, titanium, and a titanium alloy.

In order to solve the above problems, according to the present disclosure, there is provided an endoscope device including an endoscope and an endoscope imaging device that includes an attachment portion detachably connected to an eyepiece portion of the endoscope and captures a subject image emitted from the eyepiece portion,
in which at least one of the attachment portion and the endoscope imaging device includes a first portion of metal having a first sliding surface,
the endoscope imaging device includes a second portion of metal having a second sliding surface that slides on the first sliding surface, and
a resin portion is formed on at least one of the first sliding surface and the second sliding surface.

In order to solve the above problem, according to the present disclosure, there is provided an attachment device to be attached to a main body of an endoscope imaging device in which one end side is detachably connected to an eyepiece portion of an endoscope and the other end side captures a subject image emitted from the endoscope, the attachment device including:
a mount that holds the endoscope with a locking member;
a ring that is provided on an outer periphery of the mount and is capable of moving the locking member into the mount in accordance with rotation around a predetermined rotation axis; and
a resin portion configured on at least one of the mount and two sliding surfaces on which the ring slides.

### BRIEF DESCRIPTION OF DRAWINGS

FIG. 1 is a view illustrating a schematic configuration of an endoscope device according to an embodiment of the present disclosure.

FIG. 2 is an external perspective view of an endoscope imaging device.

FIG. 3 is a side view illustrating a connection portion between an endoscope and the endoscope imaging device.

FIG. 4 is a perspective view illustrating a configuration example of a mount.

FIG. 5 is a plan view of a first structure and a the ring as viewed from an arrow B side.

FIG. 6 is an enlarged view of a cross section taken along line CC in FIG. 4 in a state where a resin portion is embedded in a hole.

FIG. 7 is an external perspective view of an attachment portion.

FIG. 8 is a view illustrating a part of a DD cross section of FIG. 7.

FIG. 9 is an enlarged cross-sectional view of a sliding range in a state where the resin portion is embedded.

FIG. 10 is a view illustrating a configuration example of an endoscope device according to a second embodiment.

FIG. 11 is a perspective view illustrating an appearance of an endoscope imaging device.

FIG. 12 is a cross-sectional view of a joint portion between an imaging device main body and an attachment portion.

FIG. 13 is an enlarged cross-sectional view of a region A52 in FIG. 12 in a state where a resin portion is embedded.

### DESCRIPTION OF EMBODIMENTS

Hereinafter, embodiments of an endoscope imaging device, an endoscope device, and an attachment device will be described with reference to the drawings. Hereinafter, main components of the endoscope imaging device, the endoscope device, and the attachment device will be mainly described, but the endoscope imaging device, the endoscope device, and the attachment device may have components and functions that are not illustrated or described. The following description does not exclude components and functions that are not illustrated or described.

### First Embodiment

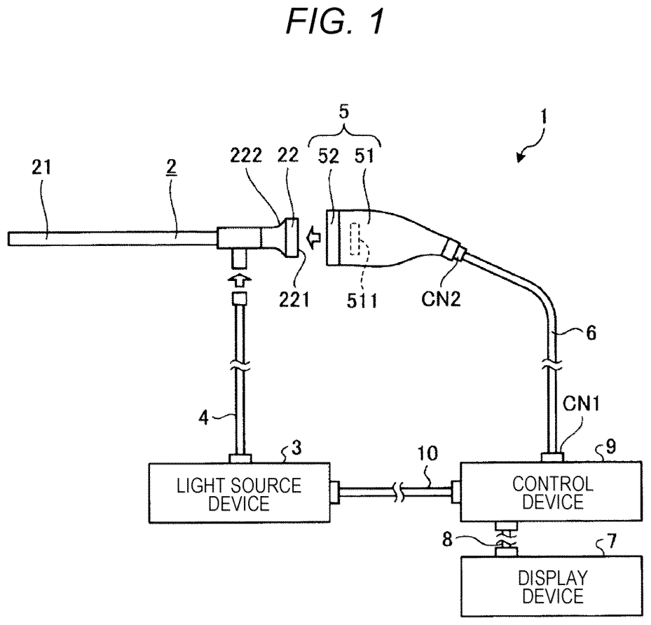

FIG. 1 is a view illustrating a schematic configuration of an endoscope device according to an embodiment of the present disclosure. The endoscope device 1 is a device that is used in the medical field and observes the inside of a living body. As illustrated in FIG. 1, the endoscope device 1 includes an endoscope 2, a light source device 3, a light guide 4, an endoscope imaging device (camera head) 5, a first transmission cable 6, a display device 7, a second transmission cable 8, a control device 9, and a third transmission cable 10.

The endoscope 2 includes a hard endoscope. That is, the endoscope 2 has an elongated shape in which the whole is hard or a part is soft and the other part is hard, and is inserted into the living body. As illustrated in FIG. 1, the endoscope 2 includes an insertion portion 21 and an eyepiece portion 22. The insertion portion 21 is a portion that extends linearly and is inserted into the living body. An optical system (not illustrated) that is configured using one or a plurality of lenses and condenses a subject image is provided in the insertion portion 21. The eyepiece portion 22 is provided at a proximal end (right end portion in FIG. 1) of the insertion portion 21. In the eyepiece portion 22, an eyepiece portion optical system (not illustrated) that emits a subject image condensed by an optical system (not illustrated) in the insertion portion 21 from the eyepiece portion 22 to the outside is provided. The diameter of the eyepiece portion 22 increases toward the proximal end side.

The light source device 3 is connected to one end of the light guide 4, and supplies light for illuminating the inside of the living body to one end of the light guide 4 under the control of the control device 9. One end of the light guide 4 is detachably connected to the light source device 3, and the other end is detachably connected to the endoscope 2. Then, the light guide 4 transmits the light supplied from the light source device 3 from one end to the other end and supplies the light to the endoscope 2. The light supplied to the endoscope 2 is emitted from the distal end (left end portion in FIG. 1) of the endoscope 2 and emitted into the living body. The light (subject image) emitted into the living body and reflected in the living body is condensed by an optical system (not illustrated) in the insertion portion 21.

The endoscope imaging device 5 includes an imaging device main body 51 (FIG. 1) in which an imaging element 511 and the like are airtightly or watertightly housed, and an attachment portion (coupler) 52 provided in the imaging device main body 51 and detachably connected to the eyepiece portion 22 of the endoscope 2. Then, under the control of the control device 9, the endoscope imaging device 5 captures a subject image condensed by the endoscope 2 and outputs an image signal (RAW signal) obtained by the imaging. The image signal is, for example, an image signal of 4K or more. The imaging element 511 is provided on an optical path of light guided by the optical system of the connected endoscope 2. Note that a detailed shape of the attachment portion 52 will be described later.

One end of the first transmission cable 6 is detachably connected to the control device 9 via a connector CN1, and the other end is detachably connected to the endoscope imaging device 5 via a connector CN2. Then, the first transmission cable 6 transmits an image signal and the like output from the endoscope imaging device 5 to the control device 9, and transmits a control signal, a synchronization signal, a clock, power, and the like output from the control device 9 to the endoscope imaging device 5. Note that transmission of an image signal or the like from the endoscope imaging device 5 to the control device 9 via the first transmission cable 6 may be performed by transmitting the image signal or the like as an optical signal or an electric signal. The similarity applies to transmission of a control signal, a synchronization signal, and a clock from the control device 9 to the endoscope imaging device 5 via the first transmission cable 6.

The display device 7 is configured using a display using liquid crystal, organic electro luminescence (EL), or the like, and displays a captured image based on a video signal from the control device 9 under the control of the control device 9.

One end of the second transmission cable 8 is detachably connected to the display device 7, and the other end is detachably connected to the control device 9. Then, the second transmission cable 8 transmits the video signal processed by the control device 9 to the display device 7.

The control device 9 includes a central processing unit (CPU) and the like, and integrally controls operations of the light source device 3, the endoscope imaging device 5, and the display device 7.

For example, the control device 9 generates a video signal by performing various processes on the image signal acquired from the endoscope imaging device 5 via the first transmission cable 6, and outputs the video signal to the display device 7 via the second transmission cable 8. Then, the display device 7 displays the captured image based on the video signal. Furthermore, the control device 9 outputs a control signal and the like to the endoscope imaging device 5 and the light source device 3 via the first and third transmission cables 6 and 10.

One end of the third transmission cable 10 is detachably connected to the light source device 3, and the other end is detachably connected to the control device 9. Then, the third transmission cable 10 transmits the control signal from the control device 9 to the light source device 3.

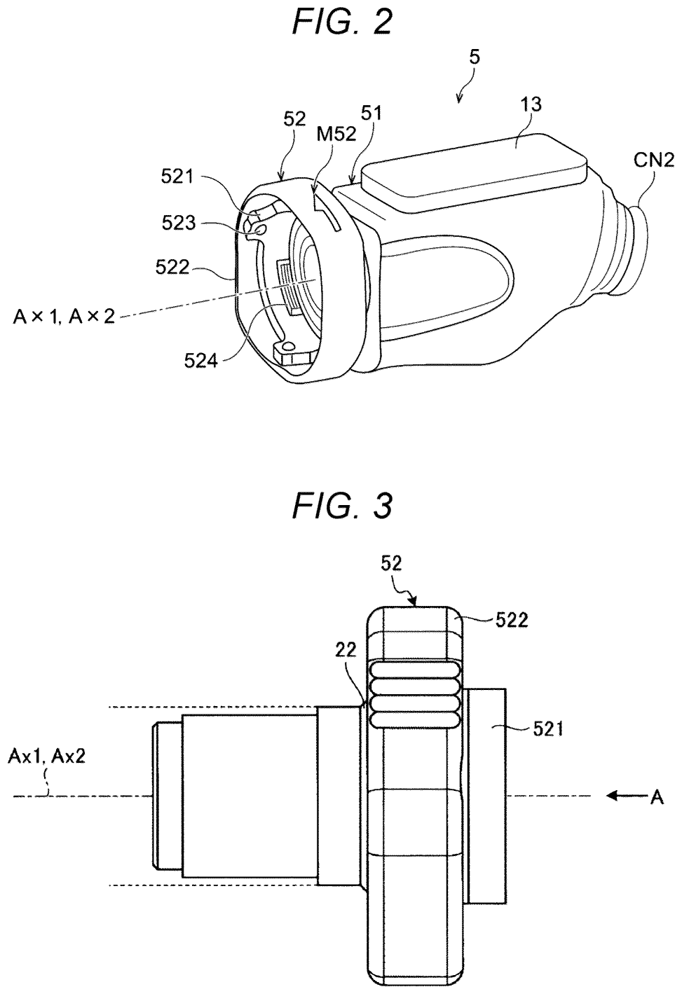

Here, a configuration of the attachment portion 52 will be described. FIG. 2 is an external perspective view of the endoscope imaging device 5. As illustrated in FIG. 2, the endoscope imaging device 5 according to the present embodiment includes an operation unit 13, the imaging device main body 51, and the attachment portion 52. FIG. 2 illustrates a state where the endoscope 2 is not attached. FIG. 3 is a side view illustrating a connection portion between the endoscope 2 and the endoscope imaging device 5. An arrow A indicates a direction from the endoscope imaging device 5 side toward the eyepiece portion 22 side.

The operation unit 13 is a portion that receives an operation by a user such as a doctor (pressed by the user). Then, the operation unit 13 outputs an operation signal corresponding to an operation on a button by the user such as a doctor to the control device 9. Note that the description will be given assuming that the central axis Ax1 of the endoscope 2 coincides with the central axis Ax2 of the endoscope imaging device 5 when the endoscope 2 is attached to the attachment portion 52. The attachment portion 52 includes a mount 521 having a substantially cylindrical shape and a ring 522 provided on the outer periphery of the mount 521 and rotatable with respect to a predetermined rotation axis. For example, the predetermined axis is the central axis Ax2 or a rotation axis qualitatively parallel to the central axis Ax2. Note that the attachment portion 52 according to the present embodiment corresponds to an attachment device. Furthermore, the rotation according to the present embodiment is referred to as rotation including movement of less than 360 degrees. For example, the rotation according to the first embodiment is a rotation of less than 360 degrees.

The mount 521 is made of, for example, a metal such as aluminum, an aluminum alloy, stainless steel, titanium, or a titanium alloy. Furthermore, the ring 522 is made of a metal such as aluminum, an aluminum alloy, stainless steel, titanium, or a titanium alloy.

In the attachment portion 52, an end surface on the distal end side (left end portion side in FIG. 2) is provided with an attachment concave portion which is recessed toward the proximal end side (right end portion side in FIG. 2) and into which the eyepiece portion 22 is inserted by the mount 521 and the ring 522. Then, in a state where the eyepiece portion 22 (see FIG. 1) is inserted into the attachment concave portion and the eyepiece portion 22 is attached to the attachment portion 52, the central axis Ax1 and the central axis Ax2 of attachment portion 52 coincide with each other. Note that details of the mount 521 and the ring 522 will be described later.

The ring 522 is provided with an inclined portion (not illustrated) that applies a biasing force to the eyepiece portion 22 to a locking member 523 (see WO 2016/103805). The thickness of the inclined portion gradually increases along the circumferential direction of the ring 522.

In the normal state, due to the elastic force acting between the mount 521 and the ring 522 in the direction opposite to a mark M52, the bottom portions of the locking members 523 are located at positions where the thickness of the inclined portion is thick. As a result, a force toward the central axis Ax2 is applied to the bottom portions of the locking members 523, and the locking members 523 protrude from the inner surface of the mount 521 toward the central axis Ax2.

On the other hand, when a user such as a doctor rotates the ring 522 in the direction of the arrow M52 against the elastic force, the bottom portions of the locking members 523 are located at positions where the thickness of the inclined portion is thin. As a result, the locking members 523 are retracted to the outer peripheral side from the central axis Ax2 and is buried in the mount 521. The form of the locking members 523 is an example, and is not limited thereto. For example, in a case where the ring 522 is rotated in the direction of the arrow M52, the locking members 523 may be deformed to such an extent that the eyepiece portion 22 can be inserted.

In addition, a hard endoscope pressing portion 524 is configured in the mount 521. The hard endoscope pressing portion 524 has a convex shape protruding toward the central axis Ax2. The side surface on the side into which the eyepiece portion 22 is inserted has a pressing surface that is a flat surface. The plane along the pressing surface is along an orthogonal line extending from the central axis Ax2 to the outer peripheral side. As illustrated in FIG. 3, in a case where the eyepiece portion 22 is attached to the attachment portion 52, a user such as a doctor rotates the ring 522 in a direction of the arrow M52 to embed the locking members 523 in the mount 521. In this state, the attachment member 221 (see FIG. 1) of the eyepiece portion 22 is brought into contact with the pressing surface of the hard endoscope pressing portion 524, and the force in the direction of the arrow M52 is relaxed. As a result, the locking members 523 protrude from the mount 521, the eyepiece portion 22 is attached to the attachment portion 52, and the locked state is obtained. In order to release the lock, a user such as a doctor may rotate the ring 522 in the direction of the arrow M52. As described above, in a state where the connection between the endoscope 2 and the endoscope imaging device 5 is locked, the endoscope imaging device 5 is not detached from the endoscope 2 by the locking members 523 provided on the mount 521. Further, when the eyepiece portion 22 is attached to the attachment portion 52, rotation of the ring 522 in a direction of the arrow M52 may cause sliding on a contact surface between the mount 521 and the ring 522. That is, the mount 521 and the ring 522 have sliding surfaces generated by relative rotation of the ring 522 with respect to the mount 521. Note that, in the present embodiment, even in a case where a resin portion is embedded in the sliding surface and sliding occurs with the other sliding surface via the resin portion of one sliding surface, the surface having the resin portion may be referred to as a sliding surface. Furthermore, in the present embodiment, three locking members 523 are provided at different positions, but at least one locking member may be provided, and the number of locking members can be arbitrarily set. Note that, for example, in a case where there are one or two locking members 523, a projection to be locked to the eyepiece portion 22 may be provided at a position different from the locking member 523. The locking member 523 only needs to be applied to at least one of the portions locked to the eyepiece portion 22.

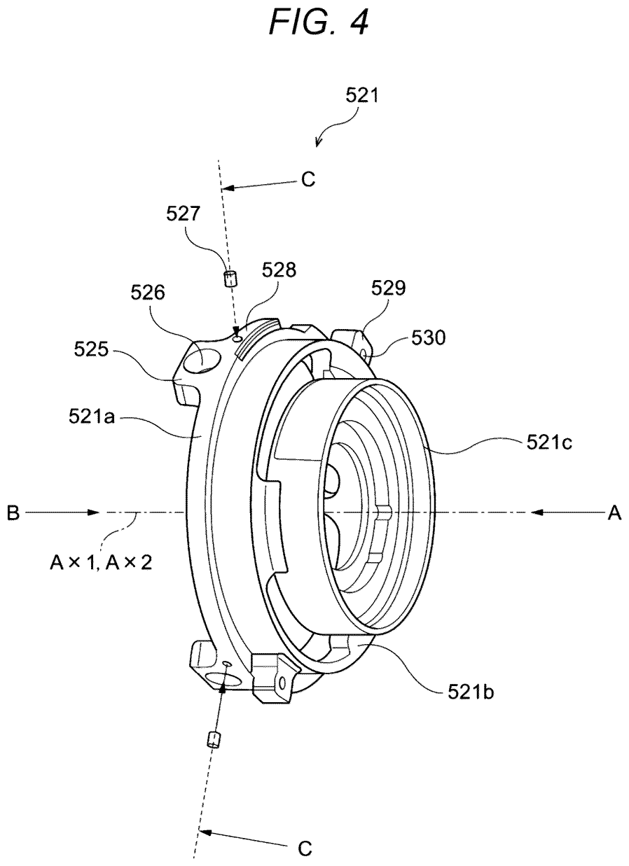

Here, a configuration example of the mount 521 will be described with reference to FIGS. 4 to 6. FIG. 4 is a perspective view illustrating a configuration example of the mount 521. An arrow A indicates a direction from the endoscope imaging device 5 side toward the eyepiece portion 22 side. An arrow B is a direction opposite to the arrow A and indicates a direction from the eyepiece portion 22 side toward the endoscope imaging device 5 side.

As illustrated in FIG. 4, the mount 521 according to the present embodiment is a structure having three stages of outer diameters with the central axis Ax2 as the central axis. The structure having the outer diameter of these stages has a substantially circular inner surface and an outer surface. For convenience of description, the structures will be referred to as a first structure 521a, a second structure 521b, and a third structure 521c in descending order of outer shapes.

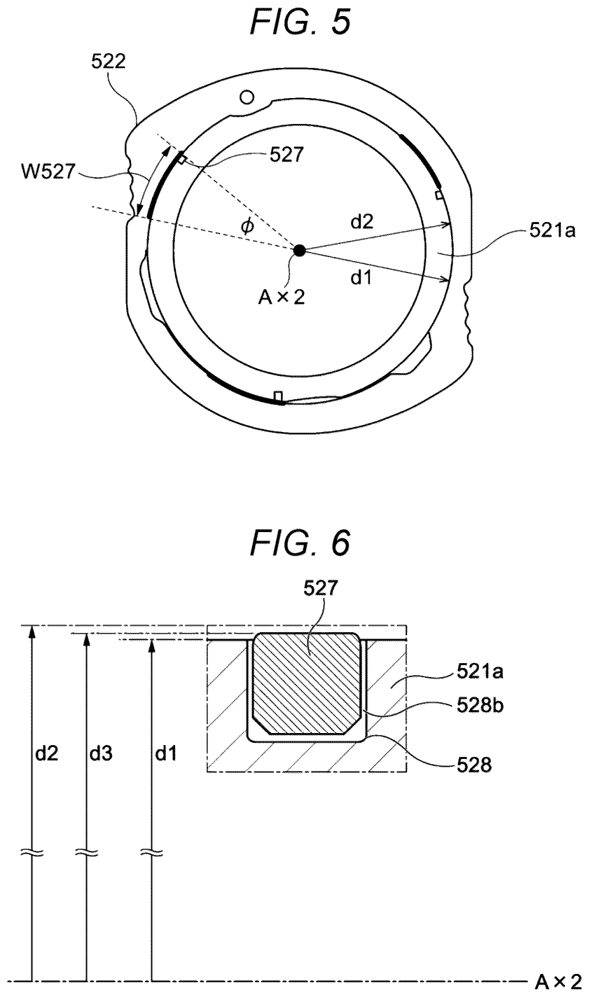

FIG. 5 is a plan view of the first structure 521a and the ring 522 as viewed from the arrow B side. As illustrated in FIG. 5, the outer surface of the first structure 521a is formed at an equal distance from the central axis Ax2 and has a surface in contact with the inner surface of the ring 522. On the other hand, the inner surface of the ring 522 is formed at an equal distance from the central axis Ax2 and has a surface in contact with the outer surface of the first structure 521a. A first radius d1 of the outer surface of the first structure 521a sliding on the inner surface of the ring 522 is smaller than a second radius d2 of the inner surface of the ring 522 sliding on the outer surface of the first structure 521a. The first radius d1 is configured in a range in which the mount 521 and the ring 522 are slidable relative to each other and the deviation from the central axis Ax2 is suppressed.

A sliding range W527 generated by the relative rotation between the mount 521 and the ring 522 is a sliding range when the eyepiece portion 22 is attached and detached.

This sliding range indicates a sliding range from a state where no force is applied to the ring 522 to a state where a force is applied in the direction of the arrow M52 (see FIG. 2) and the ring 522 rotates at the maximum rotation angle φ.

As illustrated in FIG. 4 again, the protrusion 525 has an outer surface along the inner surface of the ring 522, and holes 526 through which the locking members 523 pass are formed. Furthermore, a hole 528 in which the resin portion 527 is embedded is formed in the outer surface of the first structure 521a.

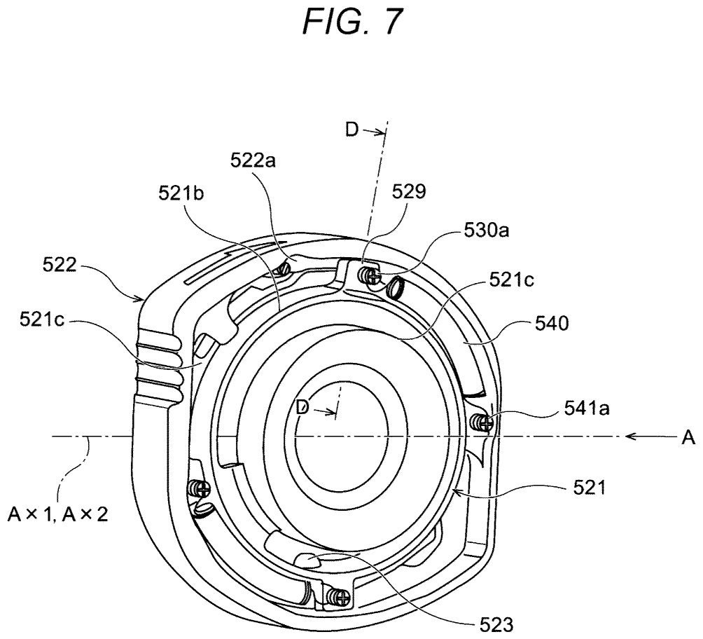

Protrusions 529 for embedding screws 530a (see FIG. 7) are formed on an outer surface of the first structure 521a. A screw hole 530 is formed in a surface of the protrusion 529 on the endoscope imaging device 5 side.

FIG. 6 is an enlarged view of the CC cross section of FIG. 4 in a state where the resin portion 527 is embedded in the hole 528. The resin portion 527 is, for example, a resin pin made of a resin material.

As illustrated in FIG. 6, a third radius d3 of the resin portion 527 in a state where the resin portion 527 is embedded in the hole 528 is a distance from the central axis Ax2 to the outer end surface of the resin portion 527. The first radius d1 of the outer surface of the first structure 521a in contact with the inner surface of the ring 522, the second radius d2 of the inner surface of the ring 522 in contact with the outer surface of the first structure 521a, and the third radius d3 to the outer end surface of the resin portion 527 have a relationship of d1<d3<d2.

As a result, in the sliding range W527 illustrated in FIG. 5, the outer end surface of the resin portion 527 comes into contact with the inner surface of the ring 522. Note that a sliding surface generated by contact between surfaces facing each other in a radial direction orthogonal to the central axis Ax2 may be referred to as a radial sliding surface, and the sliding may be referred to as radial sliding. In this way, metal-resin sliding is achieved, and the slidability of radial sliding is improved. In addition, the occurrence of metal abrasion powder on the radial sliding surface is suppressed. As a result, the occurrence of so-called rough feeling that may be caused by metal abrasion is suppressed at the time of relative rotation between the mount 521 and the ring 522. Note that the resin portion 527 can also be formed on the inner surface on the ring 522 side. In this case, the distance to the inner end surface of the resin portion 527 toward the central axis Ax2 is the third radius d3. Furthermore, the shape of the resin portion 527 may be any shape as long as sliding caused by relative rotation between the mount 521 and the ring 522 becomes metal-resin sliding.

In an embedded state where the resin portion 527 is embedded in the hole 528, a silicon material 528b is embedded in a gap between the resin portion 527 and the hole 528. As a result, the filth and the like are suppressed from remaining in the gap, and the CDS property (cleaning, disinfection, sterilization) is improved.

FIG. 7 is an external perspective view of the attachment portion 52. The attachment portion 52 is provided with elastic bodies 540. As described above, the rotation of the ring 522 with respect to the mount 521 is restricted by the biasing force of the elastic bodies 540. The elastic body 540 is, for example, a spring, and is attached to the screw 530a of the protrusion 529 formed on the mount 521 and a screw 541A attached to the ring 522. In a state where the connection between the endoscope 2 and the endoscope imaging device 5 is locked, the elastic bodies 540 are in a contracted state. In addition, movement of the mount 521 and the ring 522 in the axial direction along the central axis Ax2 is suppressed by the protrusion 529 of the mount 521 and a movement prevention portion 522a of the ring 522. Note that, in the present embodiment, a sliding surface generated by contact between the mount 521 and the ring 522 in the axial direction may be referred to as an axial sliding surface, and the sliding may be referred to as axial sliding.

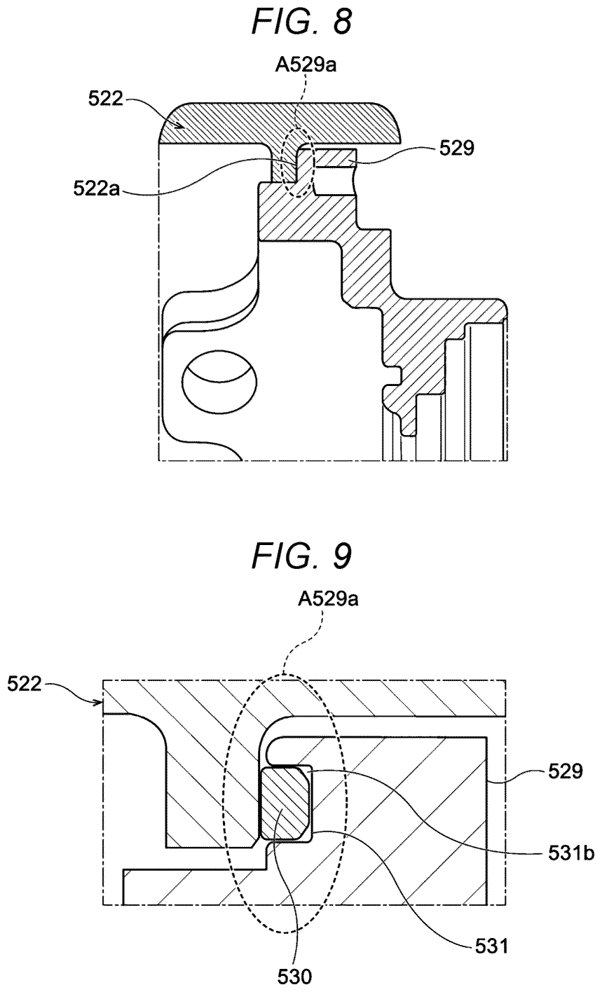

FIG. 8 is a view illustrating a part of a DD cross section of FIG. 7. As illustrated in FIG. 8, a sliding range A529a in which the side surface of the protrusion 529 on the movement prevention portion 522a side and the side surface of the movement prevention portion 522a on the protrusion 529 side axially slide is generated.

FIG. 9 is an enlarged cross-sectional view of the sliding range A529a in a state where the resin portion 530 is embedded. As illustrated in FIG. 9, the resin portion 530 is embedded in a hole 531 on the side surface of the protrusion 529 on the movement prevention portion 522a side.

As a result, the axial sliding between the side surface of the protrusion 529 on the movement prevention portion 522a side and the side surface of the movement prevention portion 522a on the protrusion 529 side becomes the axial sliding of the metal-resin, and the slidability of the axial sliding is improved. In addition, the occurrence of metal abrasion powder on the radial sliding surface is suppressed. As a result, the occurrence of so-called rough feeling that may be caused by metal abrasion is suppressed at the time of relative rotation between the mount 521 and the ring 522.

Furthermore, the resin portion 530 can also be formed on a side surface of the movement prevention portion 522a on the protrusion 529 side. Furthermore, the shape of the resin portion 527 may be any shape as long as sliding caused by relative rotation between the mount 521 and the ring 522 becomes metal-resin sliding.

In a state where the resin portion 530 is embedded in the hole 531, a silicon material 531b is embedded in a gap between the resin portion 530 and the hole 531. As a result, the filth and the like are suppressed from remaining in the gap, and the CDS property is improved.

As described above, according to the present embodiment, the resin portions 527 and 530 are formed on at least one of the sliding surfaces generated by the relative rotation between the mount 521 and the ring 522. As a result, sliding caused by relative rotation between the mount 521 and the ring 522 becomes metal-resin sliding, and the slidability is improved. In addition, the occurrence of metal abrasion powder on the sliding surfaces between the mount 521 and the ring 522 is suppressed. As a result, the occurrence of so-called rough feeling that may be caused by metal abrasion is suppressed at the time of relative rotation between the mount 521 and the ring 522.

### Second Embodiment

An endoscope imaging device 5a according to a second embodiment is different from the endoscope imaging device 5 according to the first embodiment in that the endoscope imaging device 5a is configured to be rotatable with respect to an eyepiece portion 232 of an endoscope 23 via an attachment portion (coupler) 52a. Hereinafter, differences from the endoscope imaging device 5 according to the first embodiment will be described.

### (Schematic Configuration of Endoscope System)

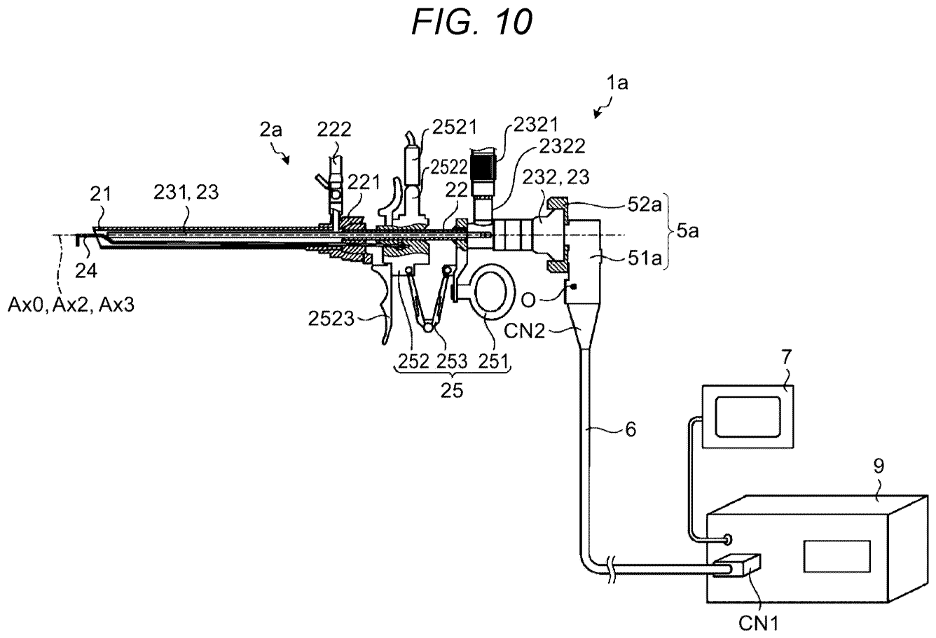

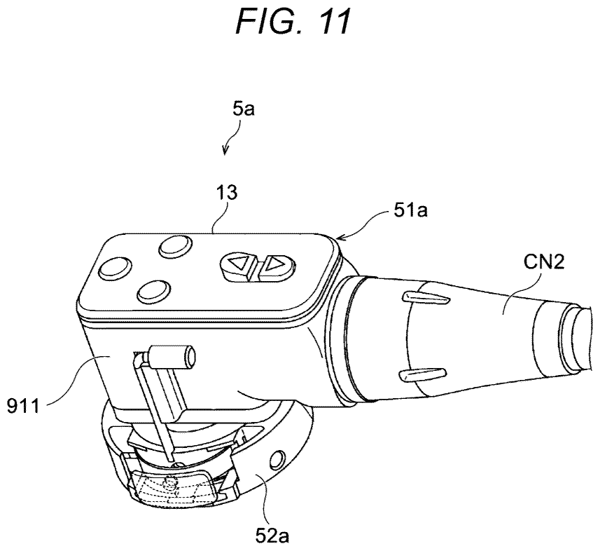

FIG. 10 is a view illustrating a configuration example of an endoscope device 1a according to the second embodiment. The endoscope device 1a is used in the medical field, and is a device that treats (incises or the like) a living tissue while observing the inside of the living body. As illustrated in FIG. 10, the endoscope device 1a includes a resectoscope 2a, an endoscope imaging device 5a, a display device 7, and a control device 9. Note that components equivalent to those of the endoscope device 1 according to the first embodiment are denoted by the same reference numerals, and description thereof may be omitted.

The resectoscope 2a is a portion that is inserted into the living body, captures a subject image, and treats a living tissue. As illustrated in FIG. 10, the resectoscope 2a includes a sheath 21, a guide tube 22, an endoscope 23, a resect electrode member 24, and a handle portion 25. The sheath 21 has a cylindrical shape and is a portion to be inserted into a living body. The guide tube 22 has an outer diameter dimension smaller than the inner diameter dimension of the sheath 21, and is inserted into the sheath 21 from the proximal end side (right side in FIG. 10) of the sheath 21. Then, the distal end side (left side in FIG. 10) of the guide tube 22 is fixed to the proximal end side of the sheath 21 via the attachment member 221 (FIG. 10). Here, the attachment member 221 is provided with a water supply port 222 for injecting liquid into the sheath 21 and supplying the liquid from a distal end (left end portion in FIG. 10) of the sheath 21.

The endoscope 23 is a portion that captures a subject image, and includes an insertion portion 231 and an eyepiece portion 232 as illustrated in FIG. 10. The insertion portion 231 is fixed in the guide tube 22 and is inserted into the sheath 21. An optical system configured using one or a plurality of lenses and condensing a subject image is provided in the insertion portion 231. The eyepiece portion 232 is connected to a proximal end (right end portion in FIG. 10) of the insertion portion 231. An eyepiece portion optical system (not illustrated) that emits a subject image condensed by the optical system in the insertion portion 231 from the eyepiece portion 232 to the outside is provided in the eyepiece portion 232. Then, the eyepiece portion 232 is formed in a tapered shape whose diameter increases toward the side (right side in FIG. 10) away from the insertion portion 231, and the endoscope imaging device 5a is detachably connected to the portion increased in diameter.

Here, as illustrated in FIG. 10, the eyepiece portion 232 is provided with a light source connector 2322 for connecting the light guide 2321. That is, the light supplied from a light source device (not illustrated) to the light guide 2321 is supplied to the insertion portion 231 via the eyepiece portion 232. The light supplied to the insertion portion 231 is emitted from the distal end (left end portion in FIG. 10) of the insertion portion 231 and emitted into the living body. The light (subject image) emitted into the living body and reflected in the living body is taken into the insertion portion 231 from the distal end of the insertion portion 231 and emitted from the eyepiece portion 232 via the optical system (not illustrated) and the eyepiece optical system (not illustrated) in the insertion portion 231.

The resect electrode member 24 is inserted into the sheath 21 via the attachment member 221, and a distal end thereof (left end portion in FIG. 10) protrudes from the distal end of the sheath 21. Then, the distal end portion of the resect electrode member 24 comes into contact with the biological tissue and treats the biological tissue with a high-frequency current. The handle portion 25 is a portion where a doctor or the like grips the resectoscope 2a and operates the resect electrode member 24. As illustrated in FIG. 10, the handle portion 25 includes a fixing ring 251, a slider 252, and a spring member 253. The fixing ring 251 is a portion on which a doctor or the like hooks a thumb, and is fixed to the guide tube 22.

The slider 252 through which the guide tube 22 is inserted is configured to be movable in the left-right direction in FIG. 10 along the guide tube 22. As illustrated in FIG. 10, the resect electrode member 24 is fixed to the slider 252. That is, the resect electrode member 24 moves forward and backward in the left and right direction in FIG. 10 in the sheath 21 as the slider 252 moves. In addition, the slider 252 is provided with a power supply connector 2522 for connecting a high frequency power supply cord 2521 connected to a high frequency power supply (not illustrated). The power supply connector 2522 is electrically connected to the resect electrode member 24 via a lead wire (not illustrated). Further, as illustrated in FIG. 10, the slider 252 is provided with a finger hook member 2523 for a doctor or the like to hook a finger other than the thumb and move the slider 252 (move the resect electrode member 24 forward and backward).

The spring member 253 has a substantially U shape, and has one end attached to the fixing ring 251 and the other end attached to the slider 252. Then, the spring member 253 biases the slider 252 toward the side away from the fixing ring 251. That is, the doctor or the like hooks the finger on the fixing ring 251 and the finger hook member 2523 and pulls the finger hook member 2523 against the biasing force of the spring member 253 to move the slider 252 to the right side in FIG. 10 (the resect electrode member 24 is moved to the right side in FIG. 10.). On the other hand, when the doctor or the like releases the finger from the finger hook member 2523, the slider 252 (resect electrode member 24) moves to the left side in FIG. 10 by the biasing force of the spring member 253.

The endoscope imaging device 5a is detachably connected to the eyepiece portion 232 of the resectoscope 2a (endoscope 23). Then, under the control of the control device 9, the endoscope imaging device 5a captures a subject image (subject image emitted from the eyepiece portion 232) captured by the endoscope 23, and outputs an image signal (RAW signal) by the imaging.

FIG. 11 is a perspective view illustrating an appearance of the endoscope imaging device 5a. As illustrated in FIG. 11, the endoscope imaging device 5a according to the present embodiment includes an operation unit 13, an imaging device main body 51a, and an attachment portion 52a. An outer case body 911 is a container-shaped member made of a metal such as aluminum, an aluminum alloy, stainless steel, titanium, or a titanium alloy.

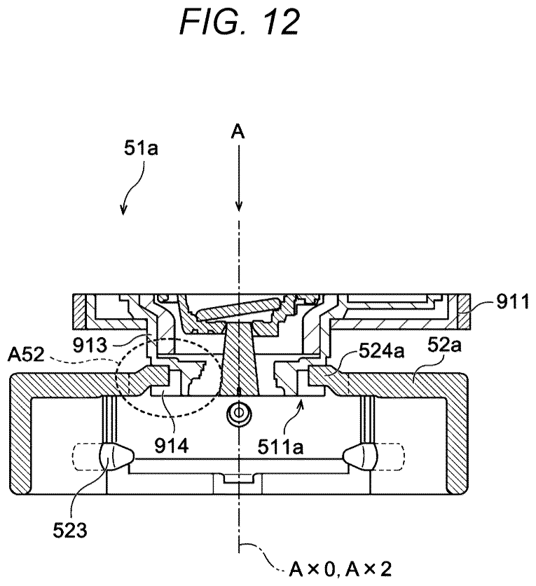

FIG. 12 is a cross-sectional view of a joint portion between the imaging device main body 51a and the attachment portion 52a. An arrow A indicates a direction from the endoscope imaging device 5 side toward the eyepiece portion 22 side. The attachment portion 52a has an annular portion 524a formed at an equal distance from a predetermined rotation axis. The rotation axis has, for example, the annular portion 524a formed at an equal distance from the optical axis of the optical axis Ax0 (Ax2) of the endoscope 23 or the rotation axis substantially parallel to the optical axis Ax0 (Ax2).

The imaging device main body 51a has a clamping portion 511a that rotatably clamps the annular portion 524a. The clamping portion 511a includes an end portion of a side wall 913 of the imaging device main body 51a and a sliding member 914. The sliding member 914 is fixed to the side wall 913 of the imaging device main body 51a on the side close to the attachment portion 52a. Note that the rotation according to the present embodiment can rotate beyond 360 degrees. That is, the imaging device main body 51a and the attachment portion 52a can relatively rotate by 360 degrees or more.

The side wall 913 of the imaging device main body 51a is made of a metal such as aluminum, an aluminum alloy, stainless steel, titanium, or a titanium alloy, and an end portion of the side wall 913 has an annular shape. The end portion of the side wall 913 has a flat surface facing the upper surface of the sliding member 914.

The sliding member 914 is made of a metal such as aluminum, an aluminum alloy, stainless steel, titanium, or a titanium alloy, and has an annular shape.

Furthermore, in the sliding member 914, the side surface facing the annular portion 524a has a flat surface facing the optical axis Ax0 of the endoscope 23. Furthermore, in the sliding member 914, the upper surface facing the lower surface of the annular portion 524a has a flat surface.

Thus, the clamping portion 511a rotatably clamps the annular portion 524a with three concave surfaces. More specifically, the flat surface of the end portion of the side wall 913 facing the upper surface of the annular portion 524a, the side surface of the sliding member 914 facing the annular portion 524a, and the upper surface of the sliding member 914 facing the lower surface of the annular portion 524a form the three concave surfaces.

As described above, the imaging device main body 51a is configured to be rotatable about the optical axis Ax0 of the endoscope 23 with respect to the endoscope 23 via the attachment portion 52a. The relative rotation between the imaging device main body 51a and the attachment portion 52a generates a sliding surface with respect to the side wall 912 and the sliding member 914 in the attachment portion 52a.

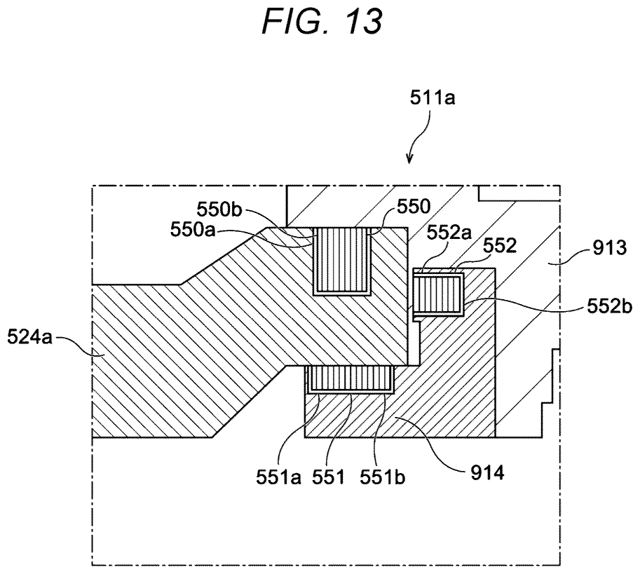

FIG. 13 is an enlarged cross-sectional view of a region A52 in FIG. 12 in a state where resin portions 550, 551, and 552 are embedded. The resin portions 550, 551, and 552 are embedded in the upper surface of the annular portion 524a, the side surface of the sliding member 914 facing the end portion of the annular portion 524a, and the upper surface of the sliding member 914 facing the lower surface of the annular portion 524a, respectively. As illustrated in FIG. 13, the resin portion 550 is embedded in a hole 550a on the upper surface of the annular portion 524a. Similarly, the resin portion 552 is embedded in a hole 552a on the side surface of the sliding member 914 facing the end portion of the annular portion 524a. Similarly, the resin portion 551 is embedded in a hole 551a on the upper surface of the sliding member 914 facing the lower surface of the annular portion 524a.

As a result, sliding between the attachment portion 52a, the side wall 912 of the imaging device main body 51a, and the sliding member 914 becomes metal-resin sliding, and the slidability is improved. Note that the resin portion 550 can also be formed on the lower surface on the movement prevention portion side wall 912 side. Similarly, the resin portion 551 can be formed on the lower surface on the attachment portion 52a side. Similarly, the resin portion 552 may be formed on the side surface on the attachment portion 52a side. Furthermore, in a state where the resin portion 550 is embedded in the hole 550a, a silicon material 550b is embedded in a gap between the resin portion 550 and the hole 550a. Similarly, in a state where the resin portion 551 is embedded in the hole 551a, a silicon material 551b is embedded in a gap between the resin portion 551 and the hole 551a.

Similarly, in a state where the resin portion 552 is embedded in the hole 552a, a silicon material 552b is embedded in a gap between the resin portion 552 and the hole 552a. As a result, the filth and the like are suppressed from remaining in the gap, and the CDS property is improved.

Note that the resin portion 550 may be configured as a flat surface of an end portion of the side wall 913 facing the upper surface of the annular portion 524a. Further, the resin portion 552 may be formed on a side surface of the annular portion 524a facing the side surface of the sliding member 914. Furthermore, the resin portion 551 may be formed on the lower surface of the annular portion 524a facing the upper surface of the sliding member 914.

As described above, the resin portions 550, 551, and 552 are formed on at least one of the sliding surfaces generated by the relative rotation between the attachment portion 52a and the imaging device main body 51a. As a result, sliding caused by relative rotation between the attachment portion 52a and the imaging device main body 51a becomes metal-resin sliding, and the slidability is improved. In addition, the occurrence of metal abrasion powder on the sliding surface between the attachment portion 52a and the imaging device main body 51a is suppressed. Accordingly, the occurrence of so-called rough feeling which may be caused by metal abrasion is suppressed at the time of relative rotation between the attachment portion 52a and the imaging device main body 51a.

Note that the present technology can have the following configurations.
(1)
An endoscope imaging device that includes an attachment portion detachably connected to an eyepiece portion of an endoscope and captures a subject image emitted from the eyepiece portion, the endoscope imaging device including:
a first portion of metal having a first sliding surface;
a second portion of metal having a second sliding surface that slides on the first sliding surface; and
a resin portion configured on at least one of the first sliding surface or the second sliding surface.
(2)
The endoscope imaging device according to (1), in which the second portion is configured to be rotatable with respect to a predetermined rotation axis.
(3)
The endoscope imaging device according to (2), in which the predetermined rotation axis is an optical axis of the endoscope or an optical axis parallel to the optical axis of the endoscope.
(4)
The endoscope imaging device according to (1), in which the resin portion is formed in a hole formed in at least one of the first sliding surface or the second sliding surface.
(5)
The endoscope imaging device according to (3), in which an adhesive is filled between the hole and the resin portion.
(6)
The endoscope imaging device according to (1), in which the attachment portion includes
the first portion, and
the second portion.
(7)
The endoscope imaging device according to (2),
in which the attachment portion includes:
a mount that holds the endoscope with a locking member; and
a ring that is provided on an outer periphery of the mount and is capable of moving the locking member into the mount in accordance with the rotation, and
the first portion is the mount, and the second portion is the ring.
(8)
The endoscope imaging device according to (7), in which at least a part of an outer peripheral surface of the mount is the first sliding surface, and at least a part of an inner peripheral surface of the ring is the second sliding surface.
(9)
The endoscope imaging device according to (8), in which a distance from the rotation axis to an end portion of the resin portion along the inner peripheral surface of the ring is larger than a distance from the rotation axis to the outer peripheral surface of the mount and shorter than a distance from the rotation axis to an end portion along the inner peripheral surface of the ring.
(10)
The endoscope imaging device according to (7),
in which the mount includes a protrusion disposed along an outer periphery of the mount and having a first side surface orthogonal to the optical axis,
the ring includes a movement prevention portion disposed along an inner peripheral surface of the ring and having a second side surface orthogonal to the optical axis and sliding on the first side surface, and
at least a part of the first side surface is the first sliding surface, and at least a part of the second side surface is the second sliding surface.
(11)
The endoscope imaging device according to (1),
in which the attachment portion is rotatably attached to an outer periphery of an opening of a main body of the endoscope imaging device, and
the first portion is the attachment portion, and the second portion is the main body.
(12)
The endoscope imaging device according to (9),
in which the attachment portion includes an annular portion formed at an equal distance from the optical axis,
the main body includes a clamping portion that clamps the annular portion, and
the resin portion is formed on a sliding surface of at least one of the annular portion and the clamping portion.
(13)
The endoscope imaging device according to (12),
in which the clamping portion clamps the annular portion with three concave surfaces, and
the resin portion is configured on at least one of the three surfaces.
(14)
The endoscope imaging device according to (1), in which the metal is at least one of aluminum, an aluminum alloy, stainless steel, titanium, or a titanium alloy.
(15)
An endoscope device including:
an endoscope; and
and an endoscope imaging device that includes an attachment portion detachably connected to an eyepiece portion of an endoscope and captures a subject image emitted from the eyepiece portion,
in which at least one of the attachment portion or the endoscope imaging device includes a first portion of metal having a first sliding surface,
the endoscope imaging device is configured to be rotatable with respect to a predetermined rotation axis and includes a second portion of metal having a second sliding surface that slides on the first sliding surface, and
a resin portion is formed on at least one of the first sliding surface and the second sliding surface.
(16)
An attachment device to be attached to a main body of an endoscope imaging device in which one end side is detachably connected to an eyepiece portion of an endoscope and the other end side captures a subject image emitted from the endoscope, the attachment device including:
a mount that holds the endoscope with a locking member;
a ring that is provided on an outer periphery of the mount and is capable of moving the locking member into the mount in accordance with rotation around a predetermined rotation axis; and
a resin portion configured on at least one of the mount or two sliding surfaces on which the ring slides.

Aspects of the present disclosure are not limited to the above-described individual embodiments, but include various modifications that can be conceived by those skilled in the art, and the effects of the present disclosure are not limited to the above-described contents. That is, various additions, modifications, and partial deletions can be made without departing from the conceptual idea and spirit of the present disclosure derived from the contents defined in the claims and equivalents thereof.

### REFERENCE SIGNS LIST

1 Endoscope device
2, 2a Endoscope
5, 5a Endoscope imaging device
51, 51a Imaging device main body
52, 52a Attachment portion (coupler)
511a Clamping portion
521 Mount
522 Ring
522a Movement prevention portion
523 Locking member, annular portion
524a, 529 Protrusion, resin portion
527, 530, 550, 551, 552 Hole
528, 531, 550a, 551a, 552a, 913 Side wall
914 Sliding member

1. An endoscope imaging device that includes an attachment portion detachably connected to an eyepiece portion of an endoscope and captures a subject image emitted from the eyepiece portion, the endoscope imaging device comprising:
a first portion of metal having a first sliding surface;
a second portion of metal having a second sliding surface that slides on the first sliding surface; and
a resin portion configured on at least one of the first sliding surface or the second sliding surface.

2. The endoscope imaging device according to claim 1, wherein the second portion is configured to be rotatable with respect to a predetermined rotation axis.

3. The endoscope imaging device according to claim 2, wherein the predetermined rotation axis is an optical axis of the endoscope or an axis parallel to the optical axis of the endoscope.

4. The endoscope imaging device according to claim 1, wherein the resin portion is formed in a hole formed in at least one of the first sliding surface or the second sliding surface.

5. The endoscope imaging device according to claim 4, wherein an adhesive is filled between the hole and the resin portion.

6. The endoscope imaging device according to claim 1, wherein the attachment portion includes
the first portion, and
the second portion.

7. The endoscope imaging device according to claim 2,
wherein the attachment portion includes
a mount that holds the endoscope with a locking member, and
a ring that is provided on an outer periphery of the mount and is capable of moving the locking member into the mount in accordance with the rotation, and
the first portion is the mount, and the second portion is the ring.

8. The endoscope imaging device according to claim 7, wherein at least a part of an outer peripheral surface of the mount is the first sliding surface, and at least a part of an inner peripheral surface of the ring is the second sliding surface.

9. The endoscope imaging device according to claim 8, wherein a distance from the rotation axis to an end portion of the resin portion along the inner peripheral surface of the ring is larger than a distance from the rotation axis to the outer peripheral surface of the mount and shorter than a distance from the rotation axis to an end portion along the inner peripheral surface of the ring.

10. The endoscope imaging device according to claim 7, wherein
the mount includes a protrusion disposed along an outer periphery of the mount and having a first side surface orthogonal to the rotation axis,
the ring includes a movement prevention portion disposed along an inner peripheral surface of the ring and having a second side surface orthogonal to the rotation axis and sliding on the first side surface, and
at least a part of the first side surface is the first sliding surface, and at least a part of the second side surface is the second sliding surface.

11. The endoscope imaging device according to claim 1, wherein
the attachment portion is rotatably attached to an outer periphery of an opening of a main body of the endoscope imaging device, and
the first portion is the attachment portion, and the second portion is the main body.

12. The endoscope imaging device according to claim 11,
wherein the attachment portion includes an annular portion formed at an equal distance from a predetermined rotation axis,
the main body includes a clamping portion that clamps the annular portion, and
the resin portion is formed on a sliding surface of at least one of the annular portion and the clamping portion.

13. The endoscope imaging device according to claim 12,
wherein the clamping portion clamps the annular portion with three concave surfaces, and
the resin portion is configured on at least one of the three surfaces.

14. The endoscope imaging device according to claim 1, wherein the metal is at least one of aluminum, an aluminum alloy, stainless steel, titanium, or a titanium alloy.

15. An endoscope device comprising:
an endoscope; and
an endoscope imaging device that includes an attachment portion detachably connected to an eyepiece portion of an endoscope and captures a subject image emitted from the eyepiece portion,
wherein at least one of the attachment portion or the endoscope imaging device includes a first portion of metal having a first sliding surface,
the endoscope imaging device includes a second portion of metal having a second sliding surface that slides on the first sliding surface, and
a resin portion is formed on at least one of the first sliding surface and the second sliding surface.

16. An attachment device to be attached to a main body of an endoscope imaging device in which one end side is detachably connected to an eyepiece portion of an endoscope and the other end side captures a subject image emitted from the endoscope, the attachment device comprising:
a mount that holds the endoscope with a locking member;
a ring that is provided on an outer periphery of the mount and is capable of moving the locking member into the mount in accordance with rotation around a predetermined rotation axis; and
a resin portion configured on at least one of the mount or two sliding surfaces on which the ring slides.
